(19) United States
(12) Patent Application Publication
(10) Pub. No.: US 2005/0222600 A1
(43) Pub. Date: Oct. 6, 2005

(54) Inverted capsular punch and arthroscopic method

(76) Inventors: Ken Yamaguchi, St. Louis, MO (US); Peter J. Dreyfuss, Naples, FL (US); Robert M. Weber, Chino Hills, CA (US)

Correspondence Address: DICKSTEIN SHAPIRO MORIN & OSHINSKY LLP, 2101 L Street, NW, Washington, DC, 20037, US

(21) Appl. No.: 11/070,098

(22) Filed: Mar. 3, 2005

Related U.S. Application Data
(60) Provisional application No. 60/549,146, filed on Mar. 3, 2004.

(51) Int. Cl. A61B 17/32

(52) U.S. Cl. 606/184

(57) ABSTRACT

An inverted capsular punch for cutting tissue. The inverted capsular punch has a punch end effector assembly comprising a fixed upper jaw and a movable lower jaw which is pivotably secured to the fixed jaw. The fixed upper jaw has a length greater than that of the movable lower jaw, and has an elongated, spoon-like configuration that allows gentle separation and retraction of the capsule from adjacent tissues and neurovascular structures in the joint. The movable lower jaw is provided with a plurality of cutting teeth or serrations that cut the capsule. The movable lower jaw is actuated by a pair of ring handles.

This application claims the benefit of U.S. Provisional Application No. 60/549,146, filed Mar. 3, 2004, the entire disclosure of which is incorporated by reference herein.

### FIELD OF THE INVENTION

The present invention relates to arthroscopic surgical methods and instruments and, more specifically, to a method and instrument for elbow arthroscopy.

### BACKGROUND OF THE INVENTION

Arthroscopic surgical instruments are used in the surgical repair of joints where the joint is not exposed by a large incision, to remove damaged cartilage or bone tissue and to repair ligament damage. Generally, arthroscopic surgical instruments include an actuating handle coupled by a tube and push rod to an end effector assembly. The end effector assembly typically includes a pair of jaws, one of which is rotatably coupled to the other, allowing therefore articulation of one jaw relative to the other. Arthroscopic cutting instruments typically include jaws which achieve a cutting action through a punch and die configuration which severs the tissue from the joint by punching the tissue with a sharp edged jaw through a die. The die typically takes the form of a fixed jaw, while the punch takes the form of a movable jaw which rotates relative to the fixed jaw from an open position to a closed position when cutting.

Arthroscopic instruments for elbow arthroscopy are inserted in the elbow joint through a small incision so that the surgeon cuts away damaged cartilage using the cutting tool while viewing the surgical site through an arthroscope. Because of the configuration of the human elbow, the surgeon needs first to cut the tight and scarred elbow capsule with a capsular punch to release the capsule from adjacent tissues and to increase the range of motion of the elbow. As the elbow capsule is closely connected to neurovascular structures present in the elbow joint (such as the brachial artery and the median nerve), it is difficult for the surgeon to lift and subsequently cut the capsule without damaging the adjacent tissues and neurovascular structures. In addition, the surgeon performing the cutting of the capsule needs to turn the punch upside down to conduct the actual cut, which is difficult to do as the finger handles of the capsular punch are also upside down in the surgeon's hand.

Accordingly, a need exists for an improved elbow capsular punch that allows separation and perforation of the capsule from adjacent tissues and neurovascular structures without accidentally damaging these tissues and structures. A capsular punch for elbow arthroscopy with a design that allows a surgeon to grip the finger handles ergonomically and to gently lift and retract the capsule from adjacent tissues and structures, without turning the punch upside down, is also desired. A need also exists for a method of conducting elbow arthroscopy by using an inverted capsular punch which does not necessitate the turning of the punch upside down within the elbow joint.

### SUMMARY OF THE INVENTION

The capsular punch of the present invention overcomes disadvantages of the prior art, such as those noted above, by providing an inverted capsular punch having a punch end effector assembly comprising a fixed upper jaw and a movable lower jaw which is pivotably secured to the fixed jaw. The fixed upper jaw has a length greater than that of the movable lower jaw, and has an elongated, spoon-like configuration that allows gentle separation and retraction of the capsule from adjacent tissues and neurovascular structures in the joint. The movable lower jaw is provided with a plurality of cutting teeth or serrations that cut the capsule. The movable lower jaw is actuated by a pair of ring handles.

The configuration of the capsular punch of the present invention allows the surgeon to effectively manipulate the instrument and to retract the capsule from the adjacent tissue without turning the punch upside down and without damaging the neurovascular structures present in the elbow joint.

Other features and advantages of the present invention will become apparent from the following description of the invention, which refers to the accompanying drawings.

### BRIEF DESCRIPTION OF THE DRAWINGS

FIG. 1 is a front view of an inverted capsular punch according to the present invention.

FIG. 2 is a partial top view of the inverted capsular punch of FIG. 1.

FIG. 3 is an enlarged perspective view of the distal end of the inverted capsular punch of FIG. 1.

FIG. 4 is a front view of the structure of FIG. 4.

FIG. 5 is an enlarged cross-sectional view of an elbow joint undergoing elbow arthroscopy.

FIGS. 6(a) and (b) are partial cross-sectional view of an elbow joint undergoing elbow arthroscopy with the inverted capsular punch of the present invention.

### DETAILED DESCRIPTION OF THE PREFERRED EMBODIMENTS

In the following detailed description, reference is made to various specific embodiments in which the invention may be practiced. These embodiments are described with sufficient detail to enable those skilled in the art to practice the invention, and it is to be understood that other embodiments may be employed, and that structural and logical changes may be made without departing from the spirit or scope of the present invention.

The term “endoscopy” encompasses arthroscopy, laparoscopy, hysteroscopy, among others, and endoscopic surgery involves the performance of surgical procedures within a patient's body through small openings as opposed to conventional open surgery through large incisions.

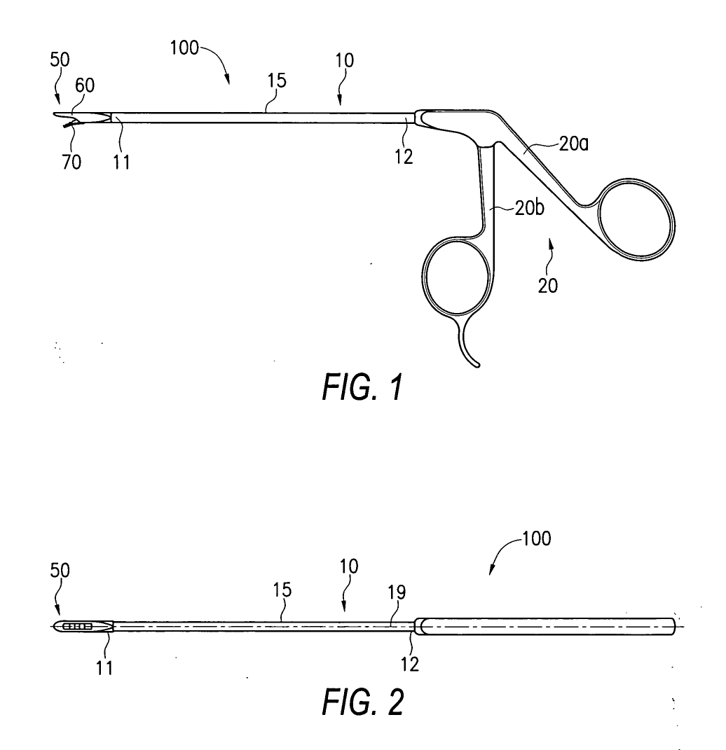

Referring now to the drawings, where like elements are designated by like reference numerals, FIGS. 1-4 illustrate an inverted capsular punch 100 of the present invention. The inverted capsular punch 100 of FIGS. 1-4 may be used in endoscopic procedures, to manipulate and cut capsular tissue during arthroscopic elbow surgery, for example.

As illustrated in FIGS. 1 and 2, the inverted capsular punch 100 includes a cannulated shaft assembly 10 provided in the shape of a cylinder or hollow tube and having a distal end 11 and a proximal end 12. The shaft assembly 10 typically has a length of about 5 cm to about 20 cm, preferably about 15 cm. The diameter of the shaft assembly is sufficiently small to facilitate introduction through access sheaths, trocars, and the like, typically being less than about 10 mm, preferably about 5 to about 7 mm, most preferably about 3.4 mm.

The shaft assembly 10 includes an elongated outer tubular member 15 which houses a coaxial push rod or inner gear member (not shown). The push rod or inner gear member has a diameter smaller than the diameter of the outer tubular member 15 and can move axially within the outer tubular member 15. As described in more detail below, the distal end 11 of the shaft is configured to engage a push end effector assembly, while the proximal end 12 of the shaft has a handle assembly designed to facilitate manual manipulation of the device and to engage and move a movable jaw member of the push end effector assembly.

The shaft or body assembly 10 of the surgical instrument 100 has a round cross-sectional shape. The shaft or body assembly 10 is formed of a rigid, medically acceptable metal or plastic material, preferably stainless steel.

In a preferred embodiment, a surgical punch end effector assembly 50 is located at the distal end 11 of the body assembly 10. The punch end effector assembly 50 comprises a fixed upper member (fixed upper jaw) 60 and a movable lower member (movable lower jaw) 70. The fixed upper member 60 is integrally attached to the outer tubular member 15 of the shaft assembly 10. The movable lower member 70 may be secured to the push rod or inner gear member of the shaft assembly 10 by a standard tip pin, for example.

Preferably, the fixed and movable jaw members 60, 70 are formed of a relatively hard material to permit smooth advancement around the neurovascular structures to be protected (when jaws 60, 70 are in the “closed” position) and yet subsequent cutting of the capsular tissue (when jaws 60, 70 are in the “open” position). The movable jaw member 70 attached to the push rod or inner gear member is actuated relative to longitudinal axis 19 of outer tubular member 15 between an “open” configuration illustrated in FIG. 1 and a “closed” configuration, by axially translating the push rod or inner gear member.

Axial translation of the push rod or inner gear member relative to the outer tubular member 15 is effected by an actuator or handle assembly 20 provided at the proximal end 12 of the body assembly 10. In one embodiment, the actuator assembly 20 may include a fixed or stationary handle 20a (stationary thumb loop 20a) and a movable lever 20b (movable finger loop 20b) connected by a pivot pin which permits the lever 20b to pivot relative to the stationary handle 20a. The movable lever 20b of the actuating assembly 20 is coupled to the push rod or inner gear member located within the body 10 such that a reciprocal axial motion is imparted to the push rod when the movable lever 20b is moved relative to the fixed handle 20a of the actuating assembly 20. The axial motion effects the opening and closing of the punch end effector assembly 50.

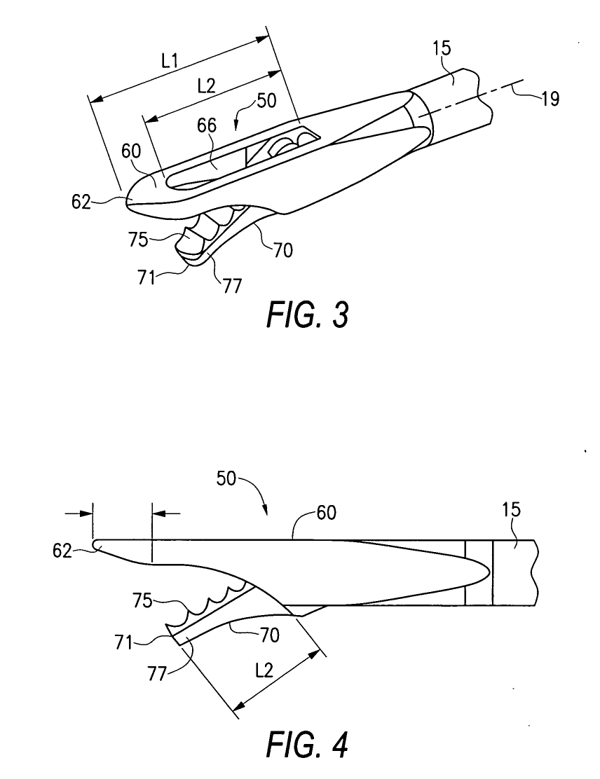

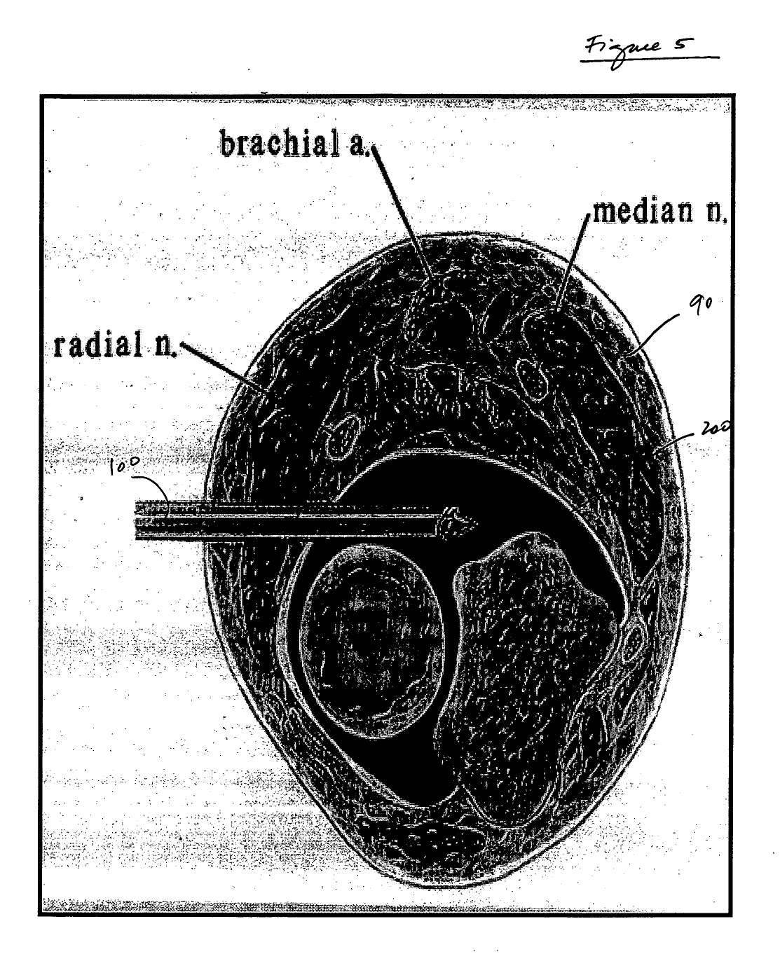

Details of the fixed upper jaw 60 and movable lower jaw 70 are illustrated in FIGS. 3 and 4. The punch end effector assembly 50 provides a punch and die cutting arrangement comprising stationary upper jaw 60 and movable lower jaw 70 which interlock. The fixed upper jaw 60 has an elongated, spoon-like configuration and is provided with a tip 62 having a substantially triangular shape terminating in a sharp tip to provide an easier penetration of the elbow capsule. The fixed upper jaw 60 is also provided with an opening 66 adapted to receive the lower movable jaw 70 closely in a tissue-cutting action. The lower movable jaw 70 has an outer periphery 77 that is adapted to be received within the opening 66 in the closed position to provide the tissue-cutting action. Length L₂ (FIG. 4) of the outer periphery 77 of the lower movable jaw 70 is about equal to length L₂ (FIG. 3) of the opening 66 of the fixed upper jaw 60 and, thus, smaller than the total length L₁ (FIG. 3) of the fixed upper jaw 60.

As also shown in FIGS. 3 and 4, a plurality of cutting teeth or serrations 75 are disposed on upper surface 71 of the lower movable jaw 70. Although the cutting teeth are shown in FIGS. 3 and 4 as having a blade-like configuration, the invention contemplates other configurations and geometries for the cutting teeth of the lower jaw.

The inverted capsular punch instrument 100 of the present invention described above with reference to FIGS. 1-4 may be employed in various surgical medical procedures for forming punctures (openings) in internal body tissue during surgical procedures. For example, the inverted capsular punch instrument 100 may be employed in endoscopic and arthroscopic procedures, including but not limited to arthroscopic elbow capsulotomy, meniscectomy (particularly for horizontal cleavage tears), shoulder capsulotomy, biceps capsulotomy, and other arthroscopic procedures that require perforation through soft tissue without damaging adjacent structures such as neurovascular structures, for example.

It will be appreciated, of course, that while the surgical instrument 100 may be particularly useful for performing remote procedures through access sheaths and trocars, it will also find use in open surgical procedures where its ability to perforate and puncture tissue will also provide advantages.

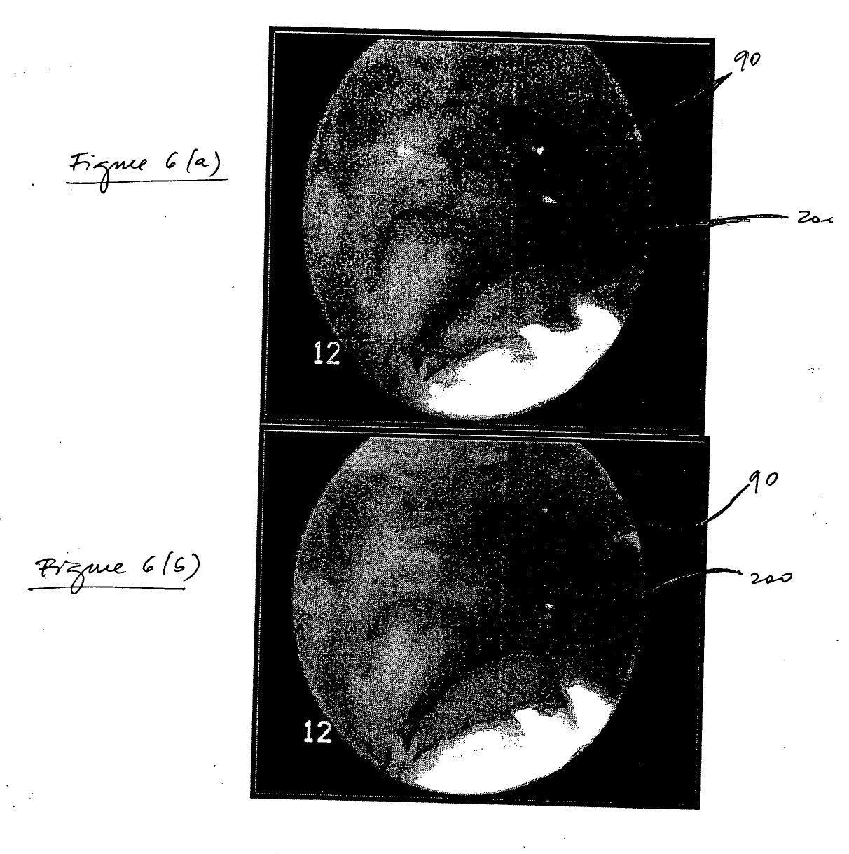

To better illustrate an exemplary surgical procedure conducted with the inverted capsular punch 100 of the present invention, reference is now made to FIGS. 5 and 6(a), (b), which illustrate a schematic view of a surgical site 90 of elbow joint 200. A surgeon advances inverted capsular punch 100 in the “straight” or closed configuration into elbow joint 200. The “straight” configuration allows the surgeon to gently lift and retract the elbow capsule tissue out of adjacent vital neurovascular structures, such as the brachial artery and the median nerve, without damaging these structures and with decreased soft tissue edema and trauma to the patient. In the “open” configuration, the surgeon perforates and cuts the capsule without turning the capsular punch 100 upside down within the joint. In this manner, the surgeon manipulates the capsular punch with increased efficiency and without damage to the adjacent elbow tissue.

The above description and drawings illustrate preferred embodiments which achieve the objects, features and advantages of the present invention. It is not intended that the present invention be limited to the illustrated embodiments. Any modification of the present invention which comes within the spirit and scope of the following claims should be considered part of the present invention.

1. An inverted arthroscopic punch instrument, comprising:
a cannulated shaft having a distal end and a proximal end;
a punch end effector assembly located at the distal end of the cannulated shaft, the punch end effector assembly comprising a fixed member having a first length and a movable member having a second length which is smaller than the first length, the fixed member being provided with an opening having a dimension that is about equal to a corresponding dimension of the movable member; and
an actuator assembly located at the proximal end of the cannulated shaft and operatively connected to the punch end effector assembly.

2. The inverted arthroscopic punch instrument of claim 1, wherein the opening of the fixed member is sized to receive the movable member when the inverted arthroscopic punch instrument is in a closed position.

3. The inverted arthroscopic punch instrument of claim 1, wherein the movable member is provided with a plurality of serrations.

4. The inverted arthroscopic punch instrument of claim 1, wherein the actuator assembly is a handle assembly comprising a stationary thumb loop and a movable finger loop.

5. The inverted arthroscopic punch instrument of claim 1, wherein the actuator assembly rotates the movable member relative to the opening of the fixed member.

6. A surgical instrument for elbow arthroscopy, comprising:
a shaft having a proximal end, a distal end and a longitudinal axis, the shaft comprising an inner member disposed coaxially within the shaft;
a fixed jaw member located at the distal end of the shaft, and integrally attached to the shaft;
a movable jaw member located at the distal end of the shaft and coupled to the inner member, the movable member being disposed in a plane below the longitudinal axis of the shaft when the surgical instrument is in an open position; and
an actuator assembly located at the proximal end of the shaft and coupled with the movable jaw member.

7. The surgical instrument of claim 6, wherein the fixed jaw member is provided with an opening that is sized to receive the movable jaw member when the surgical instrument is in a closed position.

8. The surgical instrument of claim 6, wherein the fixed jaw member has a spoon-like configuration.

9. The surgical instrument of claim 6, wherein the fixed jaw member terminates in a sharp tip.

10. The surgical instrument of claim 6, wherein the movable jaw member is provided with a plurality of teeth.

11. A method of endoscopically puncturing tissue, comprising the steps of:
providing an inverted arthroscopic punch instrument, the inverted arthroscopic punch instrument comprising a cannulated shaft having a distal end and a proximal end; a fixed jaw member located at the distal end of the shaft, the fixed jaw member being provided with an opening sized to receive a movable jaw member when the inverted arthroscopic punch instrument is in a closed position, the movable jaw member being located at the distal end of the shaft; and a handle assembly located at the proximal end of the shaft and coupled with the movable jaw member;
positioning the inverted arthroscopic punch instrument in the proximity of tissue to be punctured at a surgical site;
advancing the inverted arthroscopic punch instrument in the closed position through the tissue; and
actuating the handle assembly to move the movable jaw member from the closed position to an open position and to cut tissue at the surgical site.

12. The method of claim 11, wherein the movable jaw member is provided with a plurality of teeth that effectuate cutting of the tissue.

13. The method of claim 11, wherein the surgical site comprises elbow capsular tissue.
